(19) United States
(12) Patent Application Publication
(10) Pub. No.: US 2026/0354748 A1
(43) Pub. Date: Nov. 12, 2026

(54) METHOD FOR CONVERTING DORSAL PHOTOGRAPH INTO SPINAL RADIOGRAPH BASED ON STABLE DIFFUSION MODEL

(71) Applicants: NANJING UNIVERSITY, Nanjing (CN); NANJING DRUM TOWER HOSPITAL, Nanjing (CN)

(72) Inventors: Wujun LI, Nanjing (CN); Neng LU, Nanjing (CN); Zhong HE, Nanjing (CN); Zezhang ZHU, Nanjing (CN)

(73) Assignees: NANJING UNIVERSITY, Nanjing (CN); NANJING DRUM TOWER HOSPITAL, Nanjing (CN)

(21) Appl. No.: 19/713,906

(22) Filed: Jun. 19, 2026

Related U.S. Application Data
(63) Continuation-in-part of application No. PCT/CN2024/093581, filed on May 16, 2024.

(30) Foreign Application Priority Data
Dec. 19, 2023 (CN) 202311748637.4

(51) Int. Cl. A61B 5/00; A61B 6/00; A61H 1/02; G01T 1/00; G06T 5/60; G06T 5/70; G06T 7/00; G06T 7/13; G06T 11/00; G16H 30/40

(52) U.S. Cl. CPC A61B 5/4566; A61B 5/0077; A61B 5/4836; A61B 5/7278; A61B 6/488; A61B 6/545; A61H 1/0292; G06T 5/60; G06T 5/70; G06T 7/0014; G06T 7/13; G06T 11/00; G16H 30/40; A61B 2576/02; G01T 1/00; G06T 2207/10116; G06T 2207/20072; G06T 2207/20081; G06T 2207/20084; G06T 2207/30012; G06T 2210/41

(57) ABSTRACT

Disclosed is a method for converting a dorsal photograph into a spinal radiograph based on a stable diffusion model. A dorsal photograph is input into the stable diffusion model, during a training stage, alignment processing is performed on the dorsal photograph and the spinal radiograph, the spinal radiograph is input into an encoder to obtain a latent vector and noise is gradually added to the latent vector, a U-Net is trained to predict the noise; during an inference stage, the dorsal photograph is used as a condition, pure noise is denoised using the U-Net, and a feature vector of the dorsal photograph extracted by using a fully connected layer is mapped to an intermediate layer of the U-Net through cross attention, and finally, a Cobb angle is automatically determined using the generated spinal radiograph to predict a condition of scoliosis.

### CROSS-REFERENCE TO RELATED APPLICATIONS

This application is a continuation-in-part of International Application No. PCT/CN2024/093581, filed on May 16, 2024, which claims priority to Chinese Patent Application No. 202311748637.4, filed on Dec. 19, 2023, the entire contents of each of which are incorporated herein by reference.

### TECHNICAL FIELD

The present disclosure relates to a method for converting a dorsal photograph into a spinal radiograph based on a stable diffusion model, and belongs to the technical field of computer vision and medical image analysis.

### BACKGROUND

Deep learning is currently the most widely used artificial intelligence (AI) technology. Deep learning is capable of simulating the working mechanism of the neural network of a human brain to automatically extract useful information from a large amount of data. Medicine is a science related to human health, and a variety of complex data and problems are clinically encountered. Combining deep learning with medicine allows patients to enjoy medical services of hospitals in a community, and helps doctors to perform a rapid and accurate assessment to formulate a more efficient and safer examination and treatment plan. Deep learning has demonstrated astonishing potential and advantages in fields such as medical image analysis.

Adolescent idiopathic scoliosis (AIS) is a three-dimensional (3D) spinal deformity occurring between 10 and 18 years of age, and the cause of AIS has not been fully revealed. AIS is the most common cause affecting spinal health of adolescents and one of three major problems affecting health of primary and secondary school students in China, with an incidence rate second only to myopia and obesity. AIS not only affects the figure and self-esteem of patients, but also compresses organs, and impairs respiratory, cardiovascular, nervous and other systems, and even endangers the life of the patients. Consequently, early detection and early intervention of AIS and delaying the progression rate of scoliosis are of great clinical significance. However, in remote areas, an X-ray examination is lacking, and the patients cannot come to hospitals for regular follow-up; the X-ray examination is time-consuming and costly, has the risk of radiation exposure, and requires professional personnel to provide operation guidance. Therefore, how to reduce the reliance on the spinal X-ray examination using a low-cost, radiation-free, and easily accessible dorsal photograph is an important and difficult problem.

Most hospitals in China can only detect AIS offline. Due to unbalanced distribution of medical resources, the patients rarely find experienced orthopedic surgeons, which tends to cause a delay in diagnosis and treatment of AIS. The patient register at the hospitals, wait for consultation, and take an X-ray film, which costs time and money and increases the risk of transportation and radiation. Meanwhile, communication between the doctors and the patients is not smooth; the doctors are busy, and the patients cannot express clearly, which is prone to cause misdiagnosis, missed diagnosis, or medical disputes. If preliminary screening assessment can be performed online, the cost of the patients can be saved, efficiency of detection can be improved, and the medical level in remote areas can be greatly improved.

For conventional patent applications such as the patent No. 202110584759.9, the innovation point of the patent is that a dorsal photograph of a human body obtained can be automatically analyzed, and features of a two-dimensional (2D) dorsal photograph and 3D image information are extracted to perform classification to determine whether the scoliosis exists. However, this method has some shortcomings, for example, being not intuitive enough, and having a low classification accuracy for assessing the severity of the scoliosis. By comparison, the method of the present disclosure is more intuitive, and an X-ray image corresponding to the dorsal photograph is generated from the dorsal photograph, thereby providing more bases for determination.

An image generation technology is a technology capable of generating a new image based on input data or condition. Image generation has many applications in the medical field, such as generating an X-ray image, a magnetic resonance imaging (MRI) image, a computed tomography (CT) image, or the like, for the purposes such as auxiliary assessment, education, and research. However, image generation also faces many challenges, such as how to improve quality, diversity, consistency, and controllability of an image. Currently, some image generation models generate poor X-ray images. For example, the generated image is blurry, distorted, unnatural, or fails to meet input requirements. These models may use technologies such as generative adversarial networks (GANs) or variational autoencoders (VAEs), all of which are generation models based on a latent variable and require learning of an implicit probability distribution from input data and then sampling from the distribution to generate new data. These technologies perform well on some image generation tasks, but may encounter some difficulties on the X-ray image, such as a small data volume of the X-ray image, a complex data distribution, a large data noise, or the like.

The stable diffusion model is a new image generation technology that does not require explicitly learning a distribution of the latent variable, but gradually converts observed data into a simple prior distribution, such as Gaussian white noise, through a forward diffusion process. Then, the prior distribution is gradually restored to original data through a reverse denoising process. This process can be divided into a plurality of small steps, and each step uses a neural network to predict a denoising distribution of a next step. The stable diffusion model is capable of generating a high-quality, high-resolution, and high-diversity image, and can easily control properties of the generated image, such as a text, a category, a style, or the like. The stable diffusion model has demonstrated its potential and advantages in the fields such as medical image analysis. Whether the stable diffusion model can be improved to enable a paired input of the dorsal photograph and the X-ray image, so as to better process the noise, the detail, and the structure of the data, more flexibly adapt to the distribution and the variation of the data, and more fully utilize the input condition.

Therefore, it is desirable to provide a method of the stable diffusion model based on feature mapping, which can better process the noise, the detail, and the structure, more flexibly adapt to the distribution and the variation of the data, and display the X-ray image more intuitively.

### SUMMARY

One or more embodiments of the present disclosure provide a method for converting a dorsal photograph into a spinal radiograph based on a stable diffusion model. The method comprises: step 1: inputting the dorsal photograph provided by a user, and ensuring clarity and integrity of a dorsum; step 2: inputting the dorsal photograph as a prompt into the stable diffusion model, wherein the stable diffusion model includes an encoder, a decoder, and a U-Net, and is capable of converting the dorsal photograph into the spinal radiograph; step 3: during a model training process, performing alignment processing on the dorsal photograph and the spinal radiograph such that a posture and a shape of the dorsal photograph are consistent with a posture and a shape of the spinal radiograph; step 4: inputting an aligned spinal radiograph into the encoder to obtain a latent vector, gradually adding noise to the latent vector, and mapping a noisy latent vector to an isotropic Gaussian distribution; step 5: extracting features of the dorsal photograph using a fully connected layer, and mapping the dorsal photograph into a vector space; step 6: denoising pure noise using the U-Net, and simultaneously mapping a feature vector of the dorsal photograph to an intermediate layer of the U-Net via cross attention; step 7: inputting denoised latent vector into the decoder to generate a spinal radiograph, and predicting a condition of scoliosis; and step 8: automatically calculating a Cobb angle using the generated spinal radiograph, and comparing the Cobb angle with a Cobb angle of an actual spinal radiograph to evaluate effectiveness and accuracy of the method.

The technical solution provided by the present disclosure has the following technical effects:
1. Using the stable diffusion model can utilize the conditional input to guide the generation process, ensure authenticity and consistency of the image, and generate the corresponding X-ray image from the dorsal photograph. Compared with models such as pix2pix and CycleGAN, the stable diffusion model can better preserve details and contrast of the image, such that the generated X-ray image is clearer and closer to an actual X-ray image. In this way, more information and basis can be provided for assessment of an AIS condition, helping a doctor determine whether the patient's spine has the abnormality of scoliosis.
2. It allows a patient to obtain professional medical services at home, saving time and financial costs of the patient for seeing the doctor, and the doctor can remotely preview the X-ray image of the patient through a network platform. This method not only facilitates AIS patients, but also reduces the pressure on the hospital and improves the medical efficiency. Furthermore, the doctor can communicate and consult with other experts through the network platform to obtain more professional opinions and suggestions, thereby improving the quality and effect of treatment for the AIS patients.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numerals indicate the same structures, wherein:

FIG. 1 is a flowchart illustrating an exemplary method for converting a dorsal photograph into a spinal radiograph based on a stable diffusion model according to some embodiments of the present disclosure;

FIG. 2 is a workflow diagram according to some embodiments of the present disclosure;

FIG. 3 is a model architecture according to some embodiments of the present disclosure;

FIG. 4 is an encoder and a decoder according to some embodiments of the present disclosure;

FIG. 5 is a U-Net diagram according to some embodiments of the present disclosure;

FIG. 6 is a conditional encoder according to some embodiments of the present disclosure;

FIG. 7 is a schematic diagram illustrating an orthosis according to some embodiments of the present disclosure;

FIG. 8 is a schematic diagram illustrating a photographing device according to some embodiments of the present disclosure.

### DETAILED DESCRIPTION

In the following, the technical solutions of the embodiments of the present disclosure will be clearly and completely described with reference to the accompanying drawings. Obviously, the described embodiments are merely a portion of the embodiments of the present disclosure, and not all of the embodiments. All other embodiments obtained by a person of ordinary skill in the art based on the embodiments in the present disclosure without making inventive efforts fall within the scope of protection of the present disclosure.

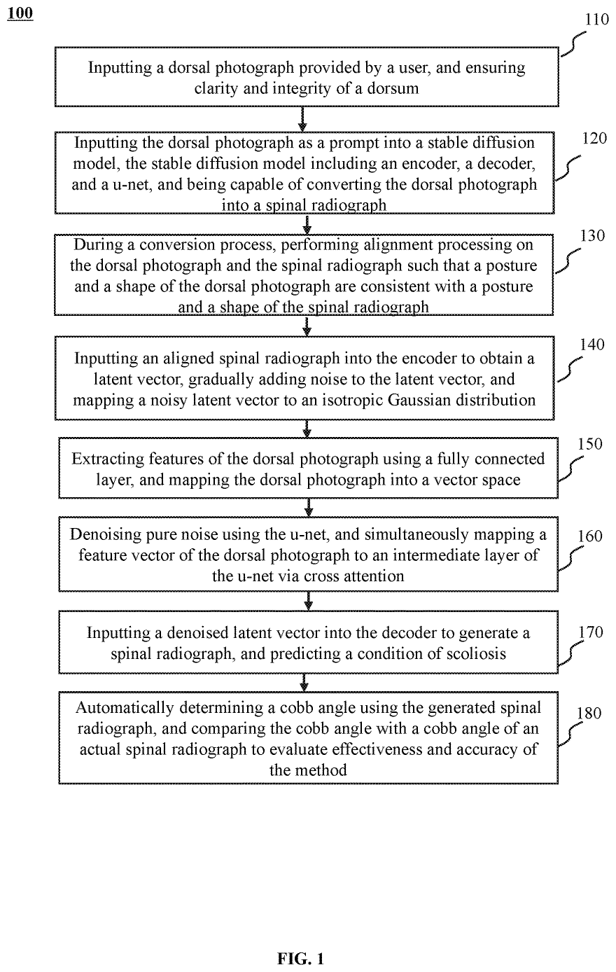

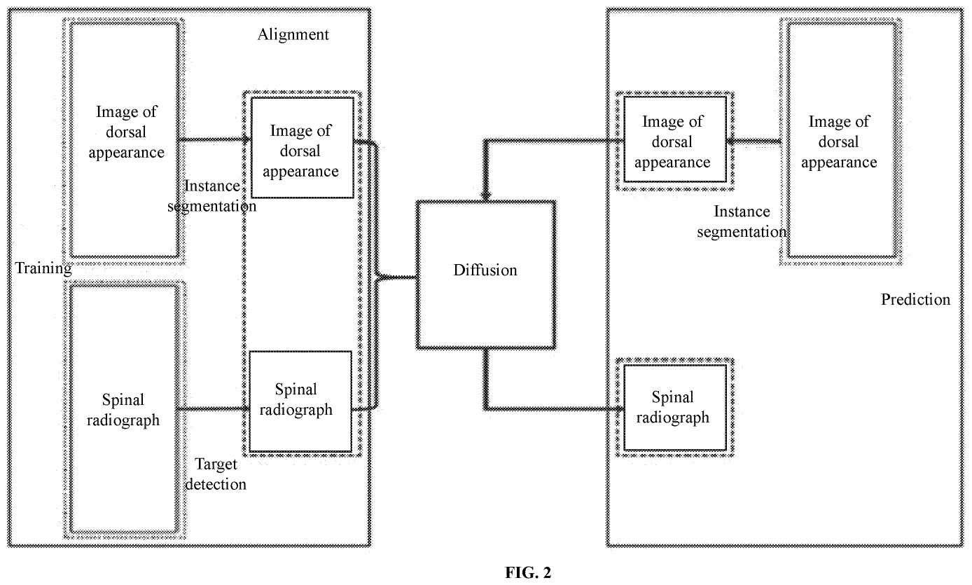

FIG. 1 is a flowchart illustrating an exemplary method for converting a dorsal photograph into a spinal radiograph based on a stable diffusion model according to some embodiments of the present disclosure.

In some embodiments, as shown in FIG. 1, a process 100 is performed by a processor. The process 100 may include steps 110 to 180.

In some embodiments, the processor may include a central processing unit (CPU), an application-specific integrated circuit (ASIC), an application-specific instruction-set processor (ASIP), a physics processing unit (PPU), a digital signal processor (DSP), a processor, a microprocessor unit, a reduced instruction set computer (RISC), a microprocessor, or the like, or any combination thereof. In some embodiments, the processor may be local or remote. In some embodiments, the processor may be implemented on a cloud platform. Merely by way of example, the cloud platform may include a private cloud, a public cloud, a hybrid cloud, a community cloud, a distributed cloud, an internal cloud, a multilayer cloud, or the like, or any combination thereof.

In 110, inputting a dorsal photograph provided by a user, and ensuring clarity and integrity of a dorsum.

The user refers to a person providing the dorsal photograph. For example, the user may be a patient.

The dorsal photograph refers to a visible light image of a dorsal appearance of a patient.

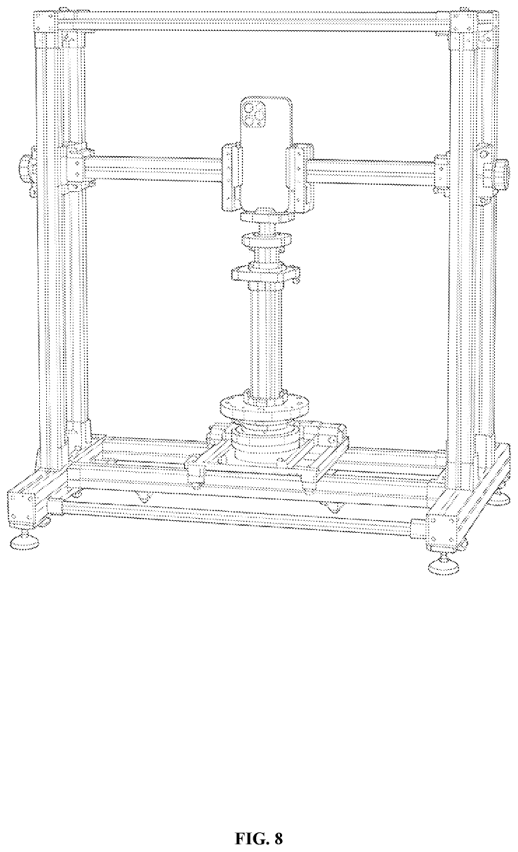

FIG. 8 is a schematic diagram illustrating a photographing device according to some embodiments of the present disclosure.

In some embodiments, as shown in FIG. 8, the user takes the dorsal photograph with an ordinary camera or a mobile phone without wearing a marker or a sensor; the user maintains an upright posture to avoid a blocked or tilted dorsum; the user takes a self-portrait or asks for assistance to take the dorsal photograph; and the dorsal photograph is clear and complete with edges and details visible, and is not blurred, dimmed, or overexposed.

The marker refers to a visible identifier artificially attached to a body or clothing, such as an optical marker point, or the like.

The sensor refers to a sensor device configured to acquire physiological or motion data, such as a pressure sensor, or the like.

In some embodiments, the present disclosure can identify a dorsal contour through pure vision and algorithms without attaching any identifier or electronic device to a patient's body, thereby completing conversion from the dorsal appearance to the spinal radiograph and scoliosis assessment.

In some embodiments of the present disclosure, by strictly defining user-side input standards, which convert professional medical image acquisition into daily photography behavior while ensuring input quality of an AI model, the present disclosure maximally reduces equipment threshold, operation threshold, safety threshold, and cost threshold for scoliosis screening, ultimately achieving the goal of low-cost, radiation-free, high-efficiency, and wide-coverage adolescent spinal health screening.

In some embodiments, the user may take a photograph of the dorsum based on a suitable photographing distance using the camera or the mobile phone to obtain a clear and complete dorsal photograph, thereby ensuring the clarity and the integrity of the dorsum in the dorsal photograph.

In 120, inputting the dorsal photograph as a prompt into a stable diffusion model, the stable diffusion model including an encoder, a decoder, and a U-Net, and being capable of converting the dorsal photograph into a spinal radiograph.

The stable diffusion model refers to a model configured to convert the dorsal photograph into the spinal radiograph. The stable diffusion model is a generative model architecture based on potential diffusion, and mainly includes an encoder, decoder, a U-Net, a conditional encoder, or the like. In some embodiments, the stable diffusion model may take the dorsal photograph as a prompt input to generate a corresponding spinal radiograph. The spinal radiograph refers to an X-ray medical image that reflects a human spine region.

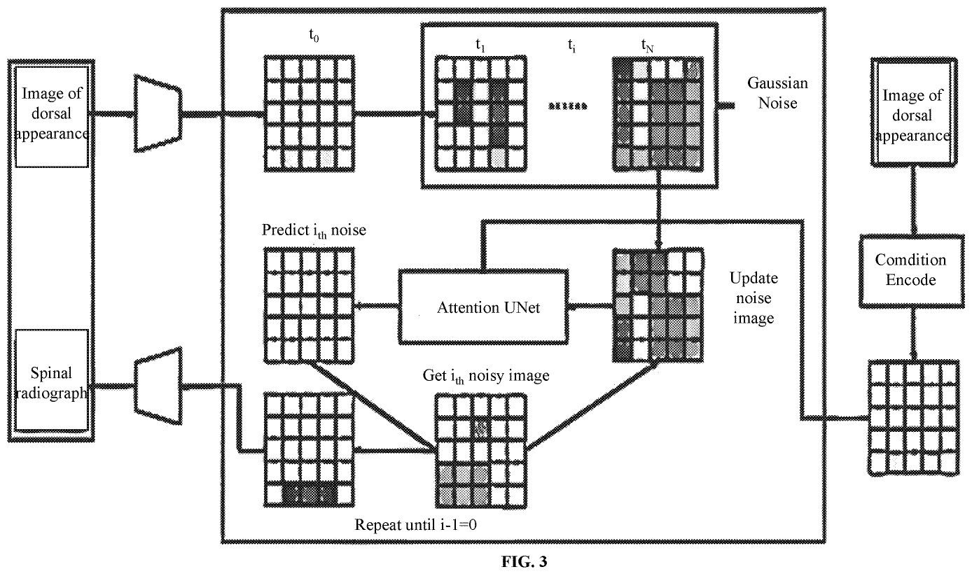

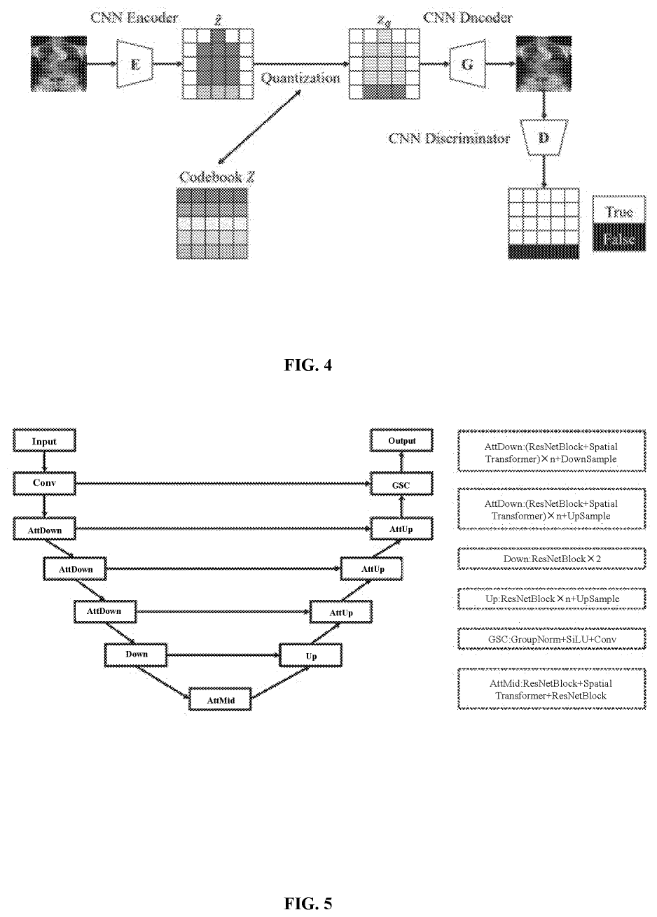

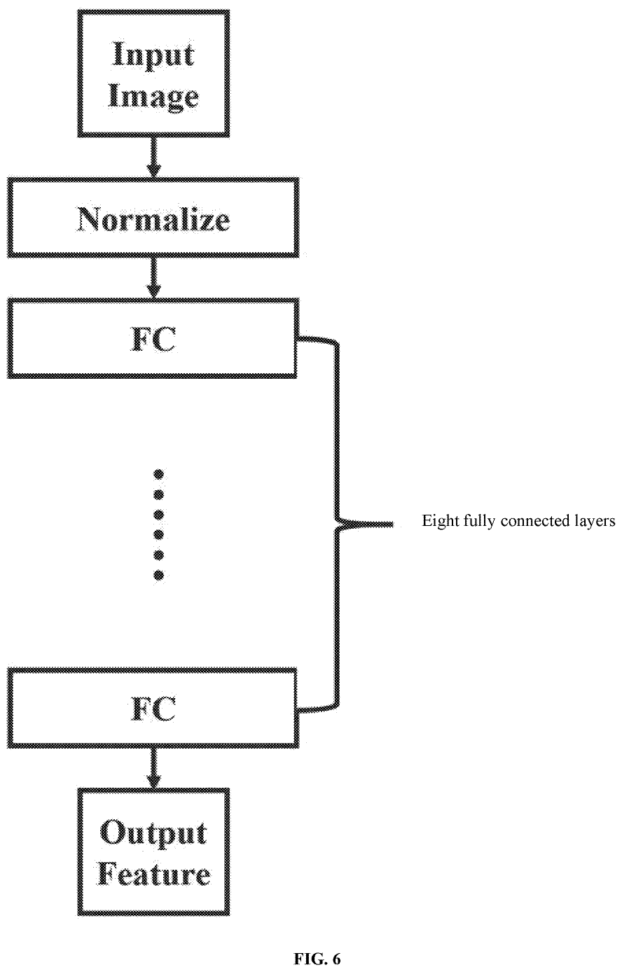

In some embodiments, as shown in FIG. 3 to FIG. 5, the stable diffusion model may include the encoder, the decoder, and the U-Net. In some embodiments, as shown in FIG. 6, the stable diffusion model may further include a conditional encoder. The conditional encoder may include a convolutional layer and a fully connected layer. The convolutional layer is configured to extract primary visual features of the dorsal photograph, and the fully connected layer is configured to synthesize and abstract convolutional features into high-level semantic feature vectors.

In some embodiments, the encoder encodes the spinal radiograph into a latent vector, and the decoder decodes the latent vector into the spinal radiograph; the U-Net is a CNN with skip connections, the U-Net denoises the latent vector and simultaneously maps features of the dorsal photograph to an intermediate layer of the U-Net via cross attention; and the stable diffusion model generates a matched spinal radiograph according to information of the dorsal photograph.

In some embodiments of the present disclosure, efficient computation is achieved through an “encoder-latent space-decoder” architecture, fidelity of medical image details is ensured through “U-Net with skip connections”, and precise and controllable guidance of the dorsal appearance for the generation process is enabled through “cross attention conditional injection”. The collaboration of the “encoder-latent space-decoder” architecture, the “U-Net with skip connections”, and the “cross attention conditional injection” allows the stable diffusion model to outperform traditional GAN/VAE solutions in terms of computational efficiency, generation quality, and conditional controllability, thereby ultimately supporting the goal of low-cost, radiation-free, and high-efficiency scoliosis preliminary screening.

In some embodiments, the stable diffusion model may be trained and obtained based on a plurality of paired first training samples. A set of first training samples may include a sample dorsal photograph and a paired actual spinal radiograph. A first training label corresponding to the set of first training samples may be actual noise added at a specific time step after a sample spinal radiograph is encoded by the encoder.

In some embodiments, the processor may obtain historically acquired paired data, including dorsal photographs of the same user and spinal radiographs corresponding to the dorsal photographs. The dorsal photographs are taken by the camera or the mobile phone, and the spinal radiographs are taken by hospital standard equipment (e.g., an X-ray machine), which together constitute the set of first training samples, where the spinal radiographs serve as a first training label source corresponding to the dorsal photographs.

In some embodiments, the processor may perform multiple iterative trainings on an initial stable diffusion model based on the plurality of paired first training samples, and ends the training when an iteration termination condition is satisfied to obtain a trained stable diffusion model. At least one of the iterative trainings includes: S1: inputting the sample spinal radiograph into a CNN Encoder to obtain a latent vector representation, and injecting Gaussian noise into the latent vector at a random time step t to obtain a noisy latent vector at the step t; S2: inputting the sample dorsal photograph into the conditional encoder (e.g., eight fully connected layers), extracting a feature vector of the dorsal photograph, and mapping the feature vector to the intermediate layer of the U-Net via a cross attention mechanism; S3: inputting the noisy latent vector at the step t into the U-Net, and combining conditional features of the dorsal photograph to obtain noise predicted by the model; S4: substituting noise predicted by the initial stable diffusion model and actual injected Gaussian noise in S1 (noise ground truth) into a predefined noise prediction loss function (e.g., a mean square error (MSE)) to calculate a value of the loss function; S5: iteratively updating model parameters of the U-Net and the conditional encoder according to the value of the loss function by gradient descent (e.g., an Adam optimizer), where the encoder and the decoder are pre-training components, and parameters of the encoder and the decoder remain frozen during the training of the diffusion model; and repeating the S1 to S5 until the training ends when the iteration termination condition is satisfied to obtain the trained stable diffusion model. The iteration termination condition may be convergence of the loss function; a count of iterations reaching a preset iteration threshold; and the value of the loss function being less than a preset function value threshold, or the like.

In some embodiments, the training of the stable diffusion model in the step 120 may further include the following steps 130 to 180.

In 130, during a conversion process, performing alignment processing on the dorsal photograph and the spinal radiograph such that a posture and a shape of the dorsal photograph are consistent with a posture and a shape of the spinal radiograph.

In some embodiments, the step 130 is performed during the model training process, the steps 130-140 are sub-steps of a model training stage, and the steps 150-180 are sub-steps of a model inference and evaluation stage.

In some embodiments, during the model training process, alignment is first performed on the dorsal photograph and the spinal radiograph to make the posture and the shape of the dorsal photograph consistent with the posture and the shape of the spinal radiograph. The purpose is to eliminate differences between two images (i.e., the dorsal photograph and the spinal radiograph) to improve accuracy and consistency of the conversion. Since the two images are taken under different conditions, there may be differences in the posture and the shape, such as angle, size, or the like. These differences may affect the conversion effect of the stable diffusion model, causing the generated spinal radiograph to be inconsistent with an actual spinal radiograph. To solve this problem, the present disclosure uses an approach of target detection (the spinal radiograph) and instance segmentation the (dorsal photograph) to automatically detect and segment key parts from the two images. This allows obtaining a set of the dorsal photograph and the spinal radiograph with consistent postures and shapes, which is used as an input and an output of the stable diffusion model.

In some embodiments of the present disclosure, by automatic alignment pre-processing, standardization of a spatial domain is completed before model processing, eliminating posture, scale, and cropping differences between the dorsal photograph and the spinal radiograph, thereby allowing the subsequent stable diffusion model to focus on learning an essential mapping relationship of “body surface appearance-bone structure” instead of being interfered by irrelevant geometric differences, and ultimately significantly improving anatomical accuracy, measurement consistency, and clinical applicability of the generated spinal radiograph.

In 140, inputting an aligned spinal radiograph into the encoder to obtain a latent vector, gradually adding noise to the latent vector, and mapping a noisy latent vector to an isotropic Gaussian distribution.

The latent vector refers to a low-dimensional continuous real-valued representation obtained by compressing and mapping high-dimensional input data (e.g., the spinal radiograph) using the encoder.

In some embodiments, the processor may obtain the latent vector in a plurality of ways.

For example, the processor performs forward propagation on the spinal radiograph using the encoder, and extracts multi-scale features of the image using multi-layer convolution and down-sampling operations, and finally maps a high-dimensional pixel space to a low-dimensional continuous latent vector representation.

The noise refers to a random variable conforming to a specific probability distribution. The specific probability distribution may be a normal distribution.

In some embodiments, the processor may add the noise to the latent vector in a plurality of ways.

For example, the processor gradually injects the noise into the latent vector within T time steps according to a preset noise scheduling strategy.

The isotropic Gaussian distribution refers to a Gaussian distribution whose covariance matrix is a multiple of an identity matrix, i.e., the noise is independently and identically distributed in each dimension with a mean of zero, equal variances, and no directional preference.

In some embodiments, the processor may map the noisy latent vector to the isotropic Gaussian distribution in a plurality of ways.

For example, as a noise adding process progresses to a final time step T, a weight of an original signal approaches zero, a noise weight approaches one, the noisy latent vector gradually loses statistical correlation with an original spinal radiograph, and finally converges to standard Gaussian white noise. At this time, the noise is independently and identically distributed in each dimension, with the mean of zero and equal variances, forming the isotropic Gaussian distribution (a pure noise state), thereby providing a simple and tractable prior starting point for a subsequent reverse denoising process. The original signal refers to structured information carried in the latent vector after being compressed by the encoder and originating from the actual spinal radiograph.

In some embodiments, the purpose of inputting the spinal radiograph into the encoder to obtain the latent vector, and gradually adding the noise to the latent vector to make the distribution the latent vector approach the isotropic Gaussian distribution is to convert observed data into a simple prior distribution in preparation for subsequent denoising and restoration; the encoder encodes the spinal radiograph into the latent vector, then the noise is gradually added to the latent vector to make the distribution of the latent vector approach the isotropic Gaussian distribution; and a complex spinal radiograph is converted into a simple noisy image as a starting point of a reverse denoising process. The starting point of the reverse denoising process is an intermediate state of the stable diffusion model.

In some embodiments of the present disclosure, by three-step conversion of “encoder compression+progressive noise addition+Gaussian prior”, a high-dimensional complex spinal radiograph generation problem is dimensionally reduced to a low-dimensional simple noise prediction problem. This conversion brings gains in four dimensions, namely computational efficiency, training stability, prior processing, and multi-scale learning, thereby enabling the stable diffusion model to be implemented from a theoretical framework as a trainable, deployable, and real-time responsive clinical screening tool, directly supporting the efficient and convenient goal of “obtaining results in less than one second” as described in the present disclosure.

In 150, extracting features of the dorsal photograph using a fully connected layer, and mapping the dorsal photograph into a vector space.

The fully connected layer refers to a layer structure in which each layer of neurons in a neural network has connection weights to all neurons in a next layer.

In some embodiments, the processor may extract the features of the dorsal photograph using the fully connected layer in a plurality of ways.

For example, the processor performs linear transformation and non-linear activation operations layer by layer on a normalized dorsal photograph through eight fully connected layers, gradually integrating and abstracting low-dimensional primary visual features into high-dimensional global semantic features, thereby extracting features comprising spinal symmetry, overall posture, and key body surface landmarks.

The vector space refers to a high-dimensional real number space formed by output features of the fully connected layer.

In some embodiments, the processor may map the dorsal photograph into the vector space in a plurality of ways.

For example, the processor projects an extracted high-dimensional feature representation into a numerical vector of fixed dimension through a last fully connected layer, causing it to be converted from an image pixel space to a structured vector space processable by a cross attention mechanism, thereby achieving the mapping of dorsal photograph information to conditional embedding.

In some embodiments, by using the fully connected layer to extract the features of the dorsal photograph and mapping the dorsal photograph into the vector space, the robustness and controllability of the conditional input are improved, thereby improving the quality of the generated spinal radiograph; the fully connected layer integrates the features extracted by a convolutional layer, thereby obtaining a high-level feature representation; and the high-level feature representation is mapped to the vector space such that the high-level feature representation is better processed and controlled.

In 160, denoising pure noise using the U-Net, and simultaneously mapping a feature vector of the dorsal photograph to an intermediate layer of the U-Net via cross attention.

The pure noise refers to a standard sampling result of the isotropic Gaussian distribution, i.e., a random vector with a mean of zero and a covariance being an identity matrix.

In some embodiments, the processor may denoise the pure noise using the U-Net in a plurality of ways.

For example, the processor may use the U-Net to predict the noise by multi-step iteration, and gradually remove random perturbations from the pure noise and restore a structured latent vector in combination with cross attention guidance of the features of the dorsal photograph.

In some embodiments, the processor may determine an offset compensation gradient based on a distribution of generated features in a latent space; perform gain adjustment on the offset compensation gradient based on an interval in which a current denoising time step falls, and inject an adjusted offset compensation gradient into the U-Net to denoise the pure noise, wherein when the current denoising time step is in a first interval, the offset compensation gradient is adjusted based on a first gain weight; when the current denoising time step is in a second interval, a principal axis direction deviation is determined based on the generated features, and a rotation correction gradient is determined based on the offset compensation gradient and the principal axis direction deviation; and the offset compensation gradient is adjusted based on the rotation correction gradient and a second gain weight; and when the current denoising time step is in a third interval, attenuation processing is performed on the offset compensation gradient to obtain the adjusted offset compensation gradient.

The generated features refer to intermediate state data in the latent space during the denoising process. For example, the generated features may be a floating point matrix [1, 4, 64, 64] representing a rough contour of spinal bones. The rough contour of the spinal bones may form an image prototype.

The distribution of generated features in the latent space refers to a numerical distribution state in each dimension of a latent tensor output by the U-Net at a specific denoising time step t in the latent space of the stable diffusion model. The latent tensor refers to a multi-dimensional floating point array obtained after the encoder compresses and maps an original image (e.g., the spinal radiograph) from a high-dimensional pixel space to a low-dimensional latent space in the stable diffusion model.

For example, assuming that the latent space is a 4×64×64 multi-dimensional space, the processor may determine a mean and a variance of the generated features in the latent space, and take the performance of the mean and the variance as the distribution of generated features in the latent space. If the distribution of the current generated features exhibits “a significant deviation of the mean from 0 ” or “a covariance matrix presenting a bar shape”, it implies that the generated spinal radiograph experiences an overall offset (e.g., overall spinal skew) in the geometric structure or an uneven energy distribution (e.g., locally overbright or overdark).

The latent space refers to a low-dimensional continuous vector space obtained by compressed mapping of high-dimensional raw data (e.g., the spinal radiograph) using the encoder.

The offset compensation gradient refers to a gradient vector configured to measure a deviation of a current sampling point from a target manifold center or an ideal Gaussian distribution path in the latent space, which is configured to correct drift in a diffusion process. The target manifold center refers to a feature aggregation center of a “normal spinal radiograph” in the latent space.

In some embodiments of the present disclosure, the processor may determine the offset compensation gradient in a plurality of ways.

For example, at each time step t, the processor may cause the U-Net to extract a current latent features zₜ, determine a probability density gradient ∇logp(zₜ) of the current latent feature, perform a differential operation on the probability density gradient with the preset target manifold center to obtain a directional derivative pointing to the target manifold center, and take the directional derivative as the offset compensation gradient.

The current denoising time step refers to an iteration number t of the stable diffusion model in a reverse diffusion process, ranging from pure Gaussian noise to a clear image. Usually, t∈[T, 0].

The interval refers to a numerical interval of the current denoising time step. For example, the interval may include the first interval, the second interval, and the third interval.

In some embodiments, the processor may divide the first interval, the second interval, and the third interval based on the current denoising time step according to a preset threshold.

For example, the processor may divide three consecutive intervals based on a numerical value of the current denoising time step t according to the preset threshold, such as the first interval (where the current denoising time step is relatively large), the second interval (where the current denoising time step is medium), and the third interval (where the current denoising time step is relatively small). The first interval corresponds to a high noise stage (macroscopic structure construction), the second interval corresponds to a medium noise stage (detail feature growth), and the third interval corresponds to a low noise stage (fine texture optimization). The preset threshold may be preset by technical personnel based on experience.

The gain adjustment refers to a dynamic weight control operation configured to amplify or reduce an amplitude of a compensation gradient. For example, the gain adjustment may be multiplying the offset compensation gradient by a coefficient of 1.5.

In some embodiments of the present disclosure, the processor may perform the gain adjustment in a plurality of ways.

For example, the processor may select a gain weight based on the interval in which the current denoising time step falls, and perform the gain adjustment based on the gain weight. The gain weight refers to a preset parameter for adjusting the offset compensation gradient. For example, the gain weight may include the first gain weight and the second gain weight, where the first interval corresponds to the first gain weight, and the second interval corresponds to the second gain weight.

Merely by way of example, if the current denoising time step is in the first interval (e.g., t∈[T, 0.7T]), an image prototype corresponding to the generated features is in a chaotic state. The adjusted offset compensation gradient may be represented by a product of the offset compensation gradient before the adjustment and the first gain weight. If the current denoising time step is in the second interval (e.g., t∈[0.7T, 0.2T]), a spinal contour appears, and the adjusted offset compensation gradient may be determined based on the offset compensation gradient before the adjustment, the rotation correction gradient, and the second gain weight. For example, when the current denoising time step is in the second interval, the adjusted offset compensation gradient may be represented by the following formula: adjusted offset compensation gradient=[(offset compensation gradient before adjustment*first preset weight)+(rotation correction gradient*second preset weight)]×second gain weight. The first preset weight and the second preset weight may be preset by technical personnel based on experience. If the current denoising time step is in the third interval (e.g., t∈[0.2T, 0]), to avoid excessively strong gradient injection causing the spinal radiograph to exhibit artificial artifacts or over-sharpening, the processor may perform exponential attenuation processing on the offset compensation gradient (e.g., reducing gain as the denoising time step decreases), thereby ensuring that the generated spinal radiograph has natural bone texture features.

In some embodiments, since the high noise stage (macroscopic structure construction) corresponding to the first interval needs to forcibly constrain an overall spinal bone structure from deviating from a normal position, the first gain weight may have a higher value (e.g., 1.5-2.0) to quickly pull back an abnormal offset of the image prototype.

In some embodiments of the present disclosure, the processor may inject the adjusted offset compensation gradient into the U-Net to denoise the pure noise.

For example, the processor may accumulate the adjusted offset compensation gradient as an additional bias term or through a feedback channel of the cross attention to the intermediate layer of the U-Net, thereby forcibly pulling back the latent vector close to the pure noise to a reasonable distribution range of the spinal structure in an initial stage of denoising, and avoiding the generated image from exhibiting an overall offset or skew.

The principal axis direction deviation refers to an angular difference between a principal feature vector direction of the latent tensor of the generated features in the latent space, after undergoing principal component analysis (PCA) or feature decomposition, and a standard vertical principal axis direction of a spinal anatomical structure. The standard vertical principal axis refers to an ideal longitudinal central axis direction that represents a normal spinal non-scoliosis state in an orthosis coordinate system or a human anatomical coordinate system, which is typically parallel to a human body gravity direction, i.e., a plumb line direction (90°).

In some embodiments, the processor may determine the principal axis direction deviation in a plurality of ways.

For example, the processor may perform PCA on an image prototype corresponding to the current generated features, extract a principal feature vector (i.e., a feature vector corresponding to a maximum eigenvalue of the image prototype obtained after the PCA), calculate an angle between the principal feature vector and the standard vertical principal axis, and determine the angle as the principal axis direction deviation.

The rotation correction gradient characterizes a partial derivative relationship generated by the principal axis direction deviation on the latent tensor.

In some embodiments, the rotation correction gradient may correct an overall tilt or rotation angle of the spinal radiograph in the generation process by fine-tuning the latent tensor without damaging spinal anatomical details.

In some embodiments of the present disclosure, the processor may determine the rotation correction gradient in a plurality of ways.

For example, the processor may set the standard vertical principal axis (e.g., 90 degrees vertical), calculate an angle Δθ (i.e., the principal axis direction deviation) between the principal feature vector direction of the current generated features and the standard vertical principal axis, calculate a partial derivative of the angle Δθ with respect to the generated features, and the partial derivative is the rotation correction gradient.

The attenuation processing refers to gradually reducing an intensity of the offset compensation gradient as the denoising time step approaches an end point (i.e., as the noise decreases and the image becomes clearer).

In some embodiments, the processor may perform the attenuation processing using a preset attenuation function (e.g., a linear attenuation function).

In some embodiments of the present disclosure, by staged gradient compensation, common structural offsets and anatomically unreasonable problems during the generation of spinal images are addressed. In an initial stage, the macroscopic distribution is ensured to be accurate; in a medium stage, a geometric structure is forcibly aligned; and in a later stage, detailed textures are protected, thereby significantly improving physiological realism of the X-ray image converted from the dorsal photograph.

The cross attention refers to a feature interaction and fusion mechanism configured to weightedly transmit the feature vector of the dorsal photograph to the U-Net to achieve dynamic guidance of conditional information on the denoising process.

The intermediate layer refers to a deep network region in the U-Net architecture located between a bottom of a down-sampling path and a top of an up-sampling path.

In some embodiments, the processor may map the feature vector of the dorsal photograph to the intermediate layer of the U-Net via the cross attention in a plurality of ways.

For example, the processor extracts the dorsal photograph into a fixed-dimensional feature vector via an 8-layer fully connected conditional encoder. The feature vector is used as a key-value input. Meanwhile, intermediate layer features of the U-Net are used as a query. The feature vector and the intermediate layer features determine a similarity weight via the cross attention, weightedly transmit the feature vector of the dorsal photograph to a deepest region of the U-Net to fuse with noise features. The noise features refer to multi-scale noise distribution information extracted by the U-Net through a down-sampling path when processing a noisy latent vector. The intermediate layer features refer to a high-semantic and low-resolution representation formed after down-sampling compression at the deepest layer (bottleneck layer/AttMid) of the U-Net architecture.

In some embodiments, the pure noise is denoised using the U-Net, and the feature vector of the dorsal photograph is simultaneously mapped to the intermediate layer of the U-Net via the cross attention. The pure noise is denoised using information of the dorsal photograph, thereby generating a matched spinal radiograph. The cross attention refers to a mechanism for implementing feature interaction and fusion, and the cross attention weightedly transmits the feature vector of the dorsal photograph to the U-Net.

In some embodiments of the present disclosure, by dynamic weighted fusion of the cross attention in the intermediate layer, the pure noise denoising capability of the U-Net is deeply coupled with conditional guidance information of the dorsal photograph, transforming the generation process from “randomly recovering any structure” to “directed reconstruction of matched spinal anatomy”. This ensures that a global posture, a scoliosis direction, and a severity of the generated X-ray image are strictly consistent with an input dorsal appearance, directly supporting the accuracy of Cobb angle calculation and the credibility of clinical screening, and is a core technical implementation link for achieving an initial screening objective of “low-cost, radiation-free, and high-efficiency”.

In 170, inputting a denoised latent vector into the decoder to generate a spinal radiograph, and predicting a condition of scoliosis.

In some embodiments, the processor may predict the condition of the scoliosis based on the spinal radiograph in a plurality of ways.

For example, the processor inputs the denoised latent vector into the decoder to reconstruct a virtual spinal radiograph, and then determines a Cobb angle of the virtual spinal radiograph; evaluates the condition according to a magnitude of the Cobb angle: wherein when the Cobb angle is greater than 10 degrees, it is evaluated as scoliosis; when the Cobb angle is greater than 20 degrees, a brace conservative treatment is recommended; and when the Cobb angle is greater than 40 degrees, a surgical treatment is recommended, thereby achieving quantitative prediction and hierarchical evaluation of the severity of the scoliosis. The Cobb angle is an indicator for evaluating the severity of the scoliosis. The Cobb angle refers to an angle determined by first identifying two most tilted vertebrae at a scoliosis site on the spinal radiograph, and then measuring an angle formed by extended lines of upper and lower endplates of the two most tilted vertebrae.

In some embodiments, the denoised latent vector is input into the decoder to generate the spinal radiograph; the denoised latent vector is restored to original data, i.e., the spinal radiograph; and a structure of the decoder is opposite to a structure of the encoder, and the decoder decodes the latent vector into the spinal radiograph.

According to some embodiments of the present disclosure, a structured latent vector denoised by the U-Net is restored to a high-resolution, high-fidelity, clinically-standard spinal radiograph through the decoder architecture symmetrical to the encoder. This converts all the AI processing steps (alignment, encoding, noise addition, denoising, and conditional injection) into a concrete medical image that is interpretable by doctors, measurable by algorithms, and understandable by patients. This directly supports automatic determination of the Cobb angle and evaluation of the scoliosis condition, and is a necessary output guarantee for achieving the goal of “low-cost, radiation-free, high-efficiency” preliminary screening.

In 180, automatically determining a Cobb angle using the generated spinal radiograph, and comparing the Cobb angle with a Cobb angle of an actual spinal radiograph to evaluate effectiveness and accuracy of the method.

In some embodiments, the processor may determine the Cobb angle using the generated spinal radiograph, and compare the Cobb angle with the Cobb angle of the actual spinal radiograph to evaluate the effectiveness and accuracy of the method. The generated spinal radiograph may be used to assist in evaluating the severity of spinal diseases such as the scoliosis. The Cobb angle is an indicator for measuring a scoliosis angle. The Cobb angle refers to an angle formed by the upper and lower endplates of the two most tilted vertebrae. The magnitude of the Cobb angle reflects the severity of the scoliosis. When the Cobb angle is greater than 10 degrees, it is evaluated as the scoliosis; when the Cobb angle is greater than 20 degrees, the brace conservative treatment is recommended; and when the Cobb angle is greater than 40 degrees, the surgical treatment is recommended.

In some embodiments, the processor may automatically detect a center of the vertebrae from the generated spinal radiograph and the actual spinal radiograph based on deep learning object detection, then determine the Cobb angle, and evaluate the effectiveness and accuracy of the method based on evaluation metrics (e.g., an MSE).

For example, the processor obtains the generated spinal radiograph and the actual spinal radiograph of the same patient, respectively. The processor automatically identifies a vertebral center and determines the Cobb angle using the same deep learning target detection. Then, the processor substitutes the generated spinal radiograph and the actual spinal radiograph into evaluation metric formulas such as the MSE and a mean absolute error (MAE) to quantify an angular deviation degree, and verifies consistency of disease classification decisions divided by 10 degree, 20 degree, and 40 degree thresholds, thereby evaluating the effectiveness and accuracy of the method.

According to some embodiments of the present disclosure, a quantitative evidence system for medical usability of generated images is established through automatic determination of the Cobb angle and systematic comparison with a true X-ray gold standard. This not only verifies the technical feasibility of a “radiation-free alternative solution”, but also directly translates the validation results into clinical decision-making language through 10°/20°/40°international grading thresholds, thereby upgrading AI-generated images from “technical demonstration” to a “diagnostic tool capable of guiding brace wearing or surgical timing”.

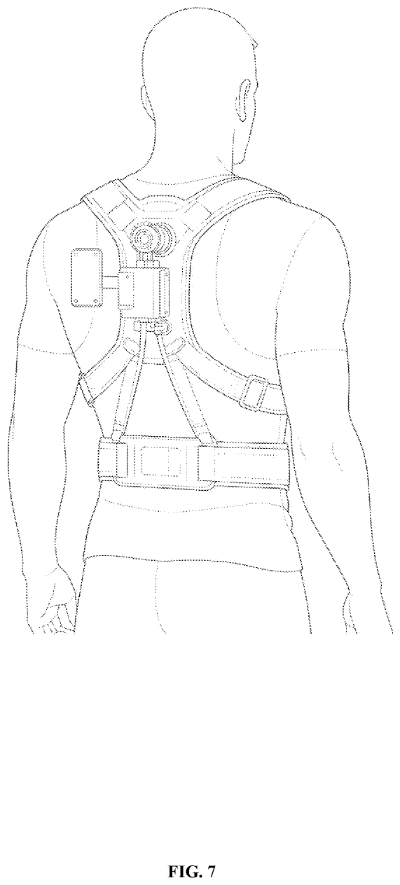

In some embodiments, as shown in FIG. 7, the processor may extract physical coordinates of a vertex of spinal curvature; determine a confidence level based on a feature map in the denoising process and the physical coordinates obtained through multiple samplings; construct a spine graph; generate a control instruction for controlling a servo motor of an orthosis based on the physical coordinates, the confidence level, and the spine graph; drive, using the control instruction, the servo motor of the orthosis to perform a rotation action to adjust a tensioning length of a tension strap of the orthosis; and during adjusting the tensioning length of the tension strap, obtain a feedback signal of the servo motor, and if the feedback signal indicates an abnormality, control the servo motor to rotate reversely to a preset initial zero position to release a compressive force of the tension strap on the dorsum.

The vertex of spinal curvature refers to a vertebral center point that is farthest from a median line in a spinal curvature curve.

In some embodiments, the vertex of spinal curvature may be a core physical point for applying a corrective force.

The physical coordinates refer to coordinates (x, y, z) that map pixel coordinates (u, v) in the spinal radiograph to a real-world centimeter scale or an orthosis hardware space coordinate system.

In some embodiments, the processor may determine the physical coordinates in a plurality of ways.

For example, the processor may use you only look once (YOLO) or a key point detection network to locate the most tilted vertebra in the generated spinal radiograph, and determine a geometric center of the most tilted vertebra; meanwhile, the processor obtains a pixel-to-distance conversion coefficient based on a ratio of a physical length to a pixel length of a known-size reference object (e.g., a standard coin) in the dorsal photograph; finally, the processor multiplies pixel coordinates of the geometric center of the most tilted vertebra by the conversion coefficient to obtain the physical coordinates in the real-world centimeter scale.

The feature map refers to a multi-dimensional response matrix generated by each layer of the U-Net, which reflects a local response intensity of the image prototype corresponding to generated features.

In some embodiments, the processor may determine the feature map in a plurality of ways.

For example, the processor may extract an activation value matrix of a last upsampling layer of the U-Net in multiple iterations as the feature map.

The multiple samplings refer to multiple denoising samplings performed on the same dorsal photograph using different random seeds. The random seeds refer to parameter values for initializing a pseudo-random number generator. In some embodiments, the random seeds may determine a specific numerical configuration of the pure noise sampled from the isotropic Gaussian distribution. For example, for each of the multiple samplings, the processor may generate different pure noise from the isotropic Gaussian distribution based on the random seeds, and obtain candidate spinal radiographs after gradual denoising by the U-Net.

In some embodiments, the processor may obtain a plurality of candidate spinal radiographs by performing the multiple samplings on the dorsal photograph, and determine a plurality of physical coordinates based on the plurality of candidate spinal radiographs.

The confidence refers to a reliability of position prediction of the vertex of spinal curvature in the generated spinal radiograph.

In some embodiments, the confidence may be a negative exponential power after adding a product of a variance of a plurality of physical coordinates and a weight factor, and a product of activation entropy and the weight factor. The activation entropy refers to an indicator for measuring a dispersion degree of a response distribution of the feature map. The weight factor refers to a dimensionless coefficient for adjusting a relative contribution ratio of a physical coordinate variance term and an activation entropy term in the confidence determination. In some embodiments, the weight factor may be preset by technical personnel based on experience.

The spine graph refers to a topological structure graph formed by spine key nodes (vertebral centers) and connecting lines between the nodes. In some embodiments, the spine graph may be configured to describe an overall curvature morphology and force trend of the spine.

In some embodiments, the nodes of the spine graph may be abstract graph nodes formed by each spine characteristic segment and a corresponding servo motor. Edges of the spine graph may be edges existing between adjacent spine segments, and edges existing between the servo motor and the spine segments on which the servo motor acts; edge features may be the Cobb angle between the spine segments.

In some embodiments, the processor may construct the spine graph in a plurality of ways.

For example, the processor may arrange a plurality of physical coordinates obtained through the multiple samplings in an anatomical order after clustering and de-duplication to form a node sequence, establish edges between adjacent nodes and determine the Cobb angle of each segment as the edge features, and establish control edges between the vertex of spinal curvature node and a corresponding servo motor, thereby forming the spine graph including a spine topological structure and an orthosis action relationship.

The orthosis refers to a treatment device for treating the scoliosis. In some embodiments, the orthosis may include a plurality of servo motors, tension straps, sensors, or the like. The servo motors may include a stepper motor, a direct current (DC) brushless servo motors, or the like, for precisely controlling a rotation angle, a rotational speed, and an output torque, thereby controlling extension and retraction of the tension straps. The tension straps may include an elastic strap, a polymer fiber strap, or the like, for transmitting a mechanical force of the servo motors to a human dorsum to form a directional corrective pressure. The sensors may include a torque sensor, a pressure sensor, or the like, for sensing a pressure distribution of the dorsum.

The rotation action refers to an angular motion output of the servo motor.

The tensioning length refers to a linear tightening amount of the tension strap generated by the rotation.

The control instruction refers to an instruction configured to control the servo motor to perform the rotation action.

In some embodiments, the processor may determine the control instruction based on the spine graph, the physical coordinates, and the confidence through an instruction generation model.

The instruction generation model refers to a model for generating the control instruction. For example, the instruction generation model may include a CNN.

In some embodiments, an input of the instruction generation model may include the spine graph, the physical coordinates, and the confidence, and an output of the instruction generation model may include the control instruction.

In some embodiments, the instruction generation model may be obtained by training based on a plurality sets of second training samples with second training labels. A set of second training samples may include a sample spine graph, sample physical coordinates, and a sample confidence. A second training label corresponding to the set of second training samples may be the control instruction corresponding to the sample spine graph, the sample physical coordinates, and the sample confidence.

In some embodiments, the processor may determine corresponding historical control instruction points as the training samples based on the sample spine graph, the sample physical coordinates, and the sample confidence in historical experiments.

In some embodiments, the processor may use an existing spinal medical image database, convert spinal medical images into a spinal skeletal model through 3D modeling, and extract a standard spine graph and physical coordinates, and use the standard spine graph and the physical coordinates as the second training samples. The processor may directly assign a high confidence label (e.g., η≈1) to the extracted spine graph and the physical coordinates, or may manually inject random offset noise (simulating possible errors) conforming to a Gaussian distribution based on the standard spine graph and the physical coordinates, and assign a low confidence label. The larger the offset of the random offset noise, the lower the manually assigned confidence η. The offset refers to a deviation between the extracted standard physical coordinates and the injected noise.

In some embodiments, the processor may use finite element analysis (FEA) and a clinical safety threshold range to set dynamic labels for samples with different confidences. The clinical safety threshold may be preset by technical personnel based on experience.

For example, the processor may establish a human torso simulation model in FEA software (e.g., ANSYS and Abaqus), simulate applying force at a spine vertex, and search for an optimal force value that can maximally correct the Cobb angle without damaging human soft tissues. The processor then maps the optimal force value to motor parameters (angle and torque) through a parameter preset table. For high confidence samples (e.g., second training samples with the confidence greater than an upper limit of the clinical safety threshold), the processor may use motor parameters corresponding to 100% of ae theoretical optimal force value as control instruction labels. For linearly transitional samples (e.g., second training samples with the confidence within the clinical safety threshold range), the processor may determine an adopted force value (e.g., the adopted force value is a product of the optimal force value and a mapping function) based on a mapping function, and use the motor parameters corresponding to the adopted force value as the control instruction labels. The mapping function may be preset by technical personnel based on the confidence. For low confidence samples (e.g., second training samples with the confidence less than a lower limit of the clinical safety threshold), the processor uses the control instruction as 5%-10% of the motor parameters corresponding to the optimal force value, which is merely for maintaining orthosis fit and does not execute large-scale compression actions, thereby ensuring safety. The parameter preset table may include a relationship between the force value and the motor parameters. The parameter preset table may be preset by technical personnel based on experience.

In some embodiments, the processor may perform multiple iterative trainings on an initial instruction generation model based on the plurality set of second training samples with the second training labels, and terminate the training when an iteration termination condition is met, thereby obtaining a trained instruction generation model. At least one of the iterative trainings includes: inputting one or more sets of second training samples into the initial instruction generation model to obtain model outputs corresponding to the one or more sets of second training samples; substituting the model outputs corresponding to the one or more sets of the second training samples, and training labels corresponding to the one or more sets of the second training samples into a formula of a pre-defined loss function to determine a value of the loss function; iteratively updating model parameters of the initial instruction generation model based on the value of the loss function, and terminating the iteration when the iteration termination condition is met, thereby obtaining the trained instruction generation model. The iterative updating of the parameters of the initial instruction generation model may be performed in a plurality of ways. For example, the iterative updating may be updated based on gradient descent. The iteration termination condition may include convergence of the or a count of iterations reaching an iteration count threshold, or the like.

The feedback signal includes a real-time current value of the motor, a torque feedback, and encoder position information. In some embodiments, the feedback signal is configured to sense physical extrusion resistance.

In some embodiments, the processor may determine whether an abnormality exists based on the feedback signal in a plurality of ways.

For example, if the current value exceeds a current threshold, it is determined that mechanical jamming exists, and an abnormality is considered to exist; if the torque feedback exceeds a torque feedback threshold, and an overrun time exceeds a time threshold, an abnormality is considered to exist; if single-step mutation of an encoder position occurs, a patient abnormal movement is considered to exist, and an abnormality is determined to exist. The current threshold, the torque feedback threshold, and the time threshold may be preset by technical personnel based on experience.

The initial zero position refers to a reference angle calibrated by the servo motor, which corresponds to a safe state where the tension strap is completely relaxed and no active pressure is applied to the dorsum.

In some embodiments, the processor may control the servo motor to rotate reversely to the initial zero position to release the compressive force.

According to some embodiments of the present disclosure, generating the control instruction and controlling the orthosis based on the control instruction achieve a closed loop from virtual image diagnosis to physical treatment. The diversity of the generation model is used to evaluate the confidence, thereby ensuring the scientific nature of the corrective force application position; the servo control and the abnormality feedback mechanism ensure the automation and safety of the correction process.

In some embodiments, the processor may extract an edge feature of a trunk contour in the dorsal photograph, and construct a force model based on the dorsal photograph; map a coordinate system of the spinal radiograph to a motor coordinate system of the orthosis based on the edge feature; determine an effective contact region between the orthosis and the dorsum based on the force model; and generate the control instruction for controlling the servo motor of the orthosis based on the physical coordinates and the confidence. The generating the control instruction includes: generating the control instruction for controlling the servo motor within an effective contact region based on the mapped motor coordinate system, the effective contact region, the physical coordinates, and the confidence.

The trunk contour refers to a dividing closed curve between a human trunk and a background in the dorsal photograph.

The edge feature refers to a marker point (e.g., an acromion process and an iliac crest projection point) having anatomical localization significance on the trunk contour and its local geometric attribute.

In some embodiments, the processor may extract the trunk contour and the edge feature of the trunk contour in a plurality of ways. For example, the processor may extract the trunk contour and the edge feature of the trunk contour through a Canny operator or a segmentation network.

The force model (biomechanical force model) refers to a simplified biomechanical model constructed based on a thickness and a width of a trunk, and a skin surface curvature in the dorsal photograph. In some embodiments, the force model may be configured to simulate spinal displacement responses under different pressures.

In some embodiments, the processor may extract a dorsal photograph contour using the Canny operator, and restore a 3D cloud map of a dorsal surface utilizing a binocular vision principle or a depth estimation model; simplify the cloud map into a mass-spring mesh model having an elastic coefficient k; and predict a force distribution of the tension strap when pressure is applied at a specific position.

The motor coordinate system refers to defining arrangement matrix coordinates of each actuator (motor) of the orthosis on a physical bracket.

In some embodiments, the processor may construct the motor coordinate system in a plurality of ways. For example, the processor may identify anatomical marker points (e.g., an acromion process and an iliac crest) in the dorsal photograph, establish an image coordinate system, and utilize an affine transformation matrix M to align internal bone physical coordinates (x, y, z)bone detected in the generated spinal radiograph with an external trunk edge feature, thereby converting to a motor array coordinate system (u, v)motor of an orthosis bracket.

The effective contact region refers to a physical region on the orthosis bracket that may effectively transmit pressure to the vertex of spinal curvature and adjacent vertebrae.

In some embodiments, the processor may determine the effective contact region in a plurality of ways.

For example, the processor may determine an effective component force generated by each contact point of the tension strap on the vertex of spinal curvature based on the force model, and determine a motor region whose resultant force vector direction points to a correction target and component force proportion exceeds 60% as the effective contact region.

In some embodiments, the processor may generate the control instruction in a plurality of ways.

For example, the processor may determine a correspondence relationship between the physical coordinates corresponding to the vertex of spinal curvature and a motor control range of the servo motor; determine whether the servo motor is located within the effective contact region; and only send the control instruction to the servo motor within the effective contact region, a control instruction intensity being positively correlated with the confidence; if the confidence is lower than the clinical safety threshold, the processor limits a maximum torque, thereby achieving precise and safe differentiated pressure output.

In some embodiments of the present disclosure, determining the control instruction based on the motor coordinate system, the effective contact region, the physical coordinates, and the confidence solves a spatial mapping problem between the virtual image and the physical device. By setting the force model and the effective contact region, the correction force can precisely act on the target region, thereby improving the wearing comfort and the correction efficiency.

In some embodiments, after comparing the Cobb angle of the generated spinal radiograph with the Cobb angle of the actual spinal radiograph to evaluate the effectiveness and the accuracy of the method, if the generated spinal radiograph meets a preset verification condition, the processor sends the spinal radiograph to an X-ray imaging device; the X-ray imaging device extracts a target region of the spinal radiograph, and drives a collimator to adjust an opening and closing state based on the physical coordinates of the target region; and predicts a bone mineral density based on pixel features of the spinal radiograph, and sets a tube voltage and a tube current-time product of the X-ray imaging device based on the bone mineral density.

The preset verification condition refers to a logical threshold that triggers physical X-ray photography of the spinal radiograph. The preset verification condition may include a determination logic based on whether the Cobb angle exceeds a severe limit value (e.g., 40 degrees), or an abnormality where a prediction value of the bone mineral density in the generated spinal radiograph is lower than a preset safety threshold (preset based on experience).

The X-ray imaging device refers to a radiographic imaging hardware entity including a high voltage generator, an X-ray tube, a digital detector, and a programmable logic controller. For example, the X-ray imaging device may be an X-ray machine.

The target region refers to an anatomical coordinate range where the scoliosis occurs, which is identified in the spinal radiograph.

In some embodiments, the processor may determine the target region in a plurality of ways.

For example, the processor may, through a control module of the X-ray imaging device, utilize a target detection algorithm (e.g., YOLOv8 based on a lightweight neural network) to automatically identify a starting vertebra and an ending vertebra of the scoliotic spine on the input generated spinal radiograph, determine minimum bounding box coordinates (x, y, w, h) of a segment including all the scoliotic spine, and determine a range defined by the bounding box coordinates as the target region.

The collimator refers to a beam limiting device provided at a front end of the X-ray tube of the X-ray imaging device. For example, the collimator may include at least two sets of mutually perpendicular lead shielding plates configured to limit a geometric shape of an X-ray beam.

The opening and closing state refers to an opening position and an opening angle of a lead plate of the collimator on a 2D plane. In some embodiments, the opening and closing state may directly determine an effective radiation field range of physical X-rays.

In some embodiments, the processor may adjust the opening and closing state in a plurality of ways.

For example, the processor may obtain a current source-to-image distance (SID) and a source-to-object distance (SOD) of the X-ray imaging device, determine a magnification factor based on a similar triangle principle, convert the pixel coordinates of the generated spinal radiograph into geometric dimensions on a physical detector plane; input the geometric dimensions into a controller of the collimator to determine physical displacements of upper, lower, left, and right lead plates inside the collimator relative to a central optical axis; and transmit a pulse instruction via a stepper motor drive bus to drive the four lead plates to move to a determined position, thereby forming a physical beam limiting range matching a geometric shape of the target region.

The pixel features refer to a grayscale mean, a grayscale standard deviation, and a texture complexity distribution of a vertebral region in the spinal radiograph. The pixel features represent a simulated feedback of a virtual tissue to an X-ray attenuation coefficient.

In some embodiments, the processor may determine the pixel features in a plurality of ways. For example, the processor may automatically locate a lumbar vertebrae 3-5 (L3-L5) region as a sampling point in the generated spinal radiograph, extract a pixel grayscale mean, a grayscale standard deviation, and a texture complexity distribution of the vertebral center within the region, and use the pixel grayscale mean, the grayscale standard deviation, and the texture complexity distribution as the pixel features.

The bone mineral density refers to a physical tissue density attribute predicted based on a deep learning model. The bone mineral density characterizes a physical quantity of an X-ray absorption capability of spinal bones.

In some embodiments, the processor may predict the bone mineral density in a plurality of ways.

For example, the processor may input the extracted pixel features into a pre-constructed bone mineral density regression model (the bone mineral density regression model is trained based on a plurality of actual spinal X-ray pixel values and corresponding dual-energy X-ray absorptiometry (DXA) bone mineral density (BMD) measurement values), and output a predicted bone mineral density value of the patient.

The tube voltage refers to a peak voltage applied to two ends of the X-ray tube. In some embodiments, the tube voltage may determine a penetration power (hardness) of the X-ray beam.

The tube current-time product refers to a product of a tube current and an exposure time. In some embodiments, the tube current-time product may determine a total photon quantity of the X-ray beam.

In some embodiments, the processor may determine the tube voltage and the tube current-time product in a plurality of ways.

For example, the processor may, based on a predicted bone mineral density and a preset image signal-to-noise ratio target, automatically retrieve an optimal combination of a corresponding tube voltage (kVp) and a tube current-time product (mAs) from an anatomically programmed radiography (APR) of a memory; directly write the retrieved tube voltage and tube current-time product values into a control register of a high voltage generator of the X-ray imaging device, thereby completing automatic presetting of physical exposure parameters. The APR may include a relationship between the BMD, an image signal-to-noise ratio, and the tube voltage and the tube current-time product. In some embodiments, the APR may be preset by a technician based on experience.

Merely by way of example, the processor may query a preset APR based on the predicted bone mineral density D. If the predicted bone mineral density is low (e.g., osteopenia), the tube voltage is automatically lowered to increase an image contrast; if the predicted bone mineral density is high (e.g., obese physique or high bone mineral density), the tube voltage is adjusted upward to ensure the penetration power, and the tube current-time product is simultaneously adjusted. The final parameters are directly written to an execution register of the high voltage generator via a bus interface of the device, thereby locking the physical exposure parameters.

In some embodiments of the present disclosure, driving the collimator to adjust the opening and closing state achieves precise beam limitation of the X-ray beam; meanwhile, the optimal combination of the tube voltage and the tube current-time product is automatically retrieved from the APR and directly written to control register of the high voltage generator, thereby completing automatic presetting of the exposure parameters, and reducing radiation exposure of non-lesion regions of the patient while ensuring that different bone quality conditions all obtain diagnostic-level image quality.

In some embodiments of the present disclosure, low-cost and high-efficiency initial screening evaluation of the scoliosis can be achieved by obtaining the spinal radiograph through the model based on the dorsal photograph without professional X-ray equipment and radiation exposure, and the safe and accessible screening tool for spinal deformities is provided by automatically determining the Cobb angle to quantify the severity of the condition.

What is claimed is:

1. A method for converting a dorsal photograph into a spinal radiograph based on a stable diffusion model, comprising:
step 1: inputting the dorsal photograph provided by a user, and ensuring clarity and integrity of a dorsum;
step 2: inputting the dorsal photograph as a prompt into the stable diffusion model, wherein the stable diffusion model includes an encoder, a decoder, and a U-Net, and is capable of converting the dorsal photograph into the spinal radiograph;
step 3: during a model training process, performing alignment processing on the dorsal photograph and the spinal radiograph such that a posture and a shape of the dorsal photograph are consistent with a posture and a shape of the spinal radiograph;
step 4: inputting an aligned spinal radiograph into the encoder to obtain a latent vector, gradually adding noise to the latent vector, and mapping a noisy latent vector to an isotropic Gaussian distribution;
step 5: extracting features of the dorsal photograph using a fully connected layer, and mapping the dorsal photograph into a vector space;
step 6: denoising pure noise using the U-Net, and simultaneously mapping a feature vector of the dorsal photograph to an intermediate layer of the U-Net via cross attention;
step 7: inputting a denoised latent vector into the decoder to generate a spinal radiograph, and predicting a condition of scoliosis; and
step 8: automatically determining a Cobb angle using the generated spinal radiograph, and comparing the Cobb angle with a Cobb angle of an actual spinal radiograph to evaluate effectiveness and accuracy of the method.

2. The method according to claim 1, wherein in the step 1, the dorsal photograph is taken by the user with an ordinary camera or a mobile phone without wearing a marker or a sensor; the user maintains an upright posture to avoid a blocked or tilted dorsum; the user takes a self-portrait or asks for assistance to take the dorsal photograph; and the dorsal photograph is clear and complete with edges and details visible, and is not blurred, dimmed, or overexposed.

3. The method according to claim 2, wherein the step 2 includes: the encoder encoding the spinal radiograph into the latent vector, and the decoder decoding the latent vector into the spinal radiograph; the U-Net being a convolutional neural network with skip connections, the U-Net denoising the latent vector and simultaneously mapping the features of the dorsal photograph to the intermediate layer of the U-Net via the cross attention; and the stable diffusion model generating a matched spinal radiograph according to information of the dorsal photograph.

4. The according to claim 3, wherein in the step 3, during a conversion process, the alignment processing is first performed on the dorsal photograph and the spinal radiograph to make the posture and the shape of the dorsal photograph consistent with the posture and the shape of the spinal radiograph, wherein the purpose of the alignment processing is to eliminate differences between the dorsal photograph and the spinal radiograph to improve accuracy and consistency of the conversion.

5. The method according to claim 4, wherein the purpose of the step 4 is to convert observed data into a simple prior distribution in preparation for subsequent denoising and restoration; the encoder encodes the spinal radiograph into the latent vector, then the noise is gradually added to the latent vector, and the latent vector is mapped to the isotropic Gaussian distribution; and the complex spinal radiograph is converted into a simple noisy image as an intermediate state of the stable diffusion model.

6. The method according to claim 5, wherein the step 5 is to improve robustness and controllability of a conditional input, thereby improving quality of the generated spinal radiograph; the fully connected layer integrates the features of the dorsal photograph extracted by a convolutional layer to obtain a high-level feature representation; and the high-level feature representation is mapped into the vector space such that the high-level feature representation is better processed and controlled.

7. The method according to claim 6, wherein the step 6 is to denoise the pure noise using the information of the dorsal photograph, thereby generating the matched spinal radiograph; and the cross attention is a mechanism for implementing feature interaction and fusion, and the cross attention transfers the feature vector of the dorsal photograph to the U-Net in a weighted manner.

8. The method according to claim 7, wherein in the step 7, the denoised latent vector is input into the decoder to generate the spinal radiograph; the denoised latent vector is restored to original data, wherein the original data is the spinal radiograph; and a structure of the decoder is opposite to a structure of the encoder, and the decoder decodes the latent vector into the spinal radiograph.

9. The method according to claim 8, wherein the step 8 is to use the generated spinal radiograph to assist in evaluating a severity of spinal diseases, wherein the spinal diseases include the scoliosis; the Cobb angle is an indicator for measuring a scoliosis angle, the Cobb angle is an angle formed by upper and lower endplates of two most tilted vertebrae; a magnitude of the Cobb angle reflects a severity of the scoliosis, wherein when the Cobb angle is greater than 10 degrees, it is evaluated as the scoliosis, when the Cobb angle is greater than 20 degrees, a brace conservative treatment is recommended; and when the Cobb angle is greater than 40 degrees, a surgical treatment is recommended.

10. The method according to claim 1, wherein the denoising pure noise using the U-Net includes:
determining an offset compensation gradient based on a distribution of generated features in a latent space;
performing gain adjustment on the offset compensation gradient based on an interval in which a current denoising time step falls, and injecting an adjusted offset compensation gradient into the U-Net to denoise the pure noise, wherein:
when the current denoising time step is in a first interval, the offset compensation gradient is adjusted based on a first gain weight;
when the current denoising time step is in a second interval, a principal axis direction deviation is determined based on the generated features, and a rotation correction gradient is determined based on the offset compensation gradient and the principal axis direction deviation; and the offset compensation gradient is adjusted based on the rotation correction gradient and a second gain weight; and
when the current denoising time step is in a third interval, attenuation processing is performed on the offset compensation gradient to obtain the adjusted offset compensation gradient.

11. The method according to claim 1, wherein after generating the spinal radiograph, the method further comprises:
extracting physical coordinates of a vertex of spinal curvature;
calculating a confidence level based on a feature map in a denoising process and the physical coordinates obtained through multiple samplings;
constructing a spine graph;
generating a control instruction for controlling a servo motor of an orthosis based on the physical coordinates, the confidence level, and the spine graph;
driving, using the control instruction, the servo motor of the orthosis to perform a rotation action to adjust a tensioning length of a tension strap of the orthosis; and
during adjusting the tensioning length of the tension strap, obtaining a feedback signal of the servo motor, and if the feedback signal indicates an abnormality, controlling the servo motor to rotate reversely to a preset initial zero position to release a compressive force of the tension strap on the dorsum.

12. The method according to claim 11, wherein before generating the control instruction for controlling the servo motor of the orthosis based on the physical coordinates and the confidence level, the method further comprises:
extracting an edge feature of a trunk contour in the dorsal photograph, and constructing a force model based on the dorsal photograph;
mapping a coordinate system of the spinal radiograph to a motor coordinate system of the orthosis based on the edge feature;
determining an effective contact region between the orthosis and the dorsum based on the force model;
the generating a control instruction for controlling the servo motor of the orthosis based on the physical coordinates and the confidence level includes:
generating the control instruction for controlling the servo motor within the effective contact region based on a mapped motor coordinate system, the effective contact region, the physical coordinates, and the confidence level.

13. The method according to claim 1, wherein after the step 8, the method further comprises:
if the generated spinal radiograph meets a preset verification condition, sending the spinal radiograph to an X-ray imaging device;
extracting, by the X-ray imaging device, a target region of the spinal radiograph, and driving a collimator to adjust an opening and closing state based on coordinates of the target region; and
predicting a bone mineral density based on pixel features of the spinal radiograph, and setting a tube voltage and a tube current-time product of the X-ray imaging device based on the bone mineral density.
